(19) United States
(12) Patent Application Publication
(10) Pub. No.: US 2008/0227825 A1
(43) Pub. Date: Sep. 18, 2008

(54) Formulation

(76) Inventors: Yatindra Joshi, Princeton, NJ (US); James Kowalski, Belle Mead, NJ (US); Jay Parthiban Lakshman, Cedar Knolls, NJ (US); Alan Edward Royce, Saylorsburg, PA (US); Wei-Qin Tong, Basking Ridge, NJ (US); Madhav Vasanthavada, Basking Ridge, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/067,346

(22) PCT Filed: Sep. 25, 2006
(86) PCT No.: PCT/US06/37198

§ 371 Date: Mar. 19, 2008

Related U.S. Application Data
(60) Provisional application No. 60/722,624, filed on Sep. 29, 2005.

(51) Int. Cl. A61K 31/4436; A61K 31/40; A61P 3/08; A61K 31/155

(52) U.S. Cl. 514/342; 514/423; 514/635

(57) ABSTRACT

This invention relates to a formulation comprising a dipeptidylpeptidase IV (DPP-IV) inhibitor preferably vildagliptin and metformin, to tablets comprising such formulations and to processes for the preparation thereof.

This invention relates to a formulation comprising a dipeptidylpeptidase IV (DPP-IV) inhibitor preferably vildagliptin and metformin, to tablets comprising such formulations and to processes for the preparation thereof.

Metformin has been widely prescribed for lowering blood glucose in patients with NIDDM and is marketed in 500, 750, 850 and 1000 mg strengths. However, because it is a short acting drug, metformin requires twice-daily or three-times-daily dosing (500-850 mg tab 2-3/day or 1000 mg bid with meals). The biguanide antihyperglycemic agent metformin disclosed in U.S. Pat. No. 3,174,901 is currently marketed in the U.S. in the form of its hydrochloride salt (Glucophage@), Bristol-Myers Squibb Company). The preparation of metformin (dimethyldiguanide) and its hydrochloride salt is state of the art and was disclosed first by Emil A. Werner and James Bell, J. Chem. Soc. 121, 1922, 1790-1794. Metformin, can be administered e.g. in the form as marketed under the trademarks GLUCOPHAGE™. Metformin, increases the sensitivity to insulin in peripheral tissues of the hosts. Metformin is also involved in inhibition of glucose absorption from the intestine, suppression of hepatic gluconeogenesis, and inhibition of fatty acid oxidation. Suitable dosage regimens of Metformin include unit doses of 500 mg two to three time's daily and can even be build up to five times daily or 850 mg once or twice daily. [Martindale, The Complete Drug Reference.

The term “metformin” as employed herein refers to metformin or a pharmaceutically acceptable salt thereof such as the hydrochloride salt, the metformin (2:1) fumarate salt, and the metformin (2:1) succinate salt as disclosed in U.S. application Ser. No. 09/262,526 filed Mar. 4, 1999, the hydrobromide salt, the p-chlorophenoxy acetate or the embonate, and other known metformin salts of mono and dibasic carboxylic acids including those disclosed in U.S. Pat. No. 3,174,901, all of which salts are collectively referred to as metformin. It is preferred that the metformin employed herein be the metformin hydrochloride salt, namely, that marketed as GLUCOPHAGE-D or GLUCOPHAGE XR (trademark of Bristol-Myers Squibb Company).

In the present context “a DPP-IV inhibitor”, “metformin”, “a glitazone”, or any specific glitazone like “pioglitazone”, “rosiglitazone”, is also intended to comprise any pharmaceutically acceptable salt thereof, crystal form, hydrate, solvate, diastereoisomer or enantiomer thereof.

The preferred DPP-IV inhibitor compounds to which this invention is primarily directed are described below:

In the present context “a DPP-IV inhibitor” is also intended to comprise active metabolites and prodrugs thereof, such as active metabolites and prodrugs of DPP-IV inhibitors. A “metabolite” is an active derivative of a DPP-IV inhibitor produced when the DPP-IV inhibitor is metabolised. A “prodrug” is a compound that is either metabolised to a DPP-IV inhibitor or is metabolised to the same metabolite(s) as a DPP-IV inhibitor.

DPP-IV inhibitors are known in the art. For example, DPP-IV inhibitors are in each case generically and specifically disclosed e.g. in WO 98/19998, DE19616 486 A1, WO 00/34241, WO 95/15309, WO 01/72290, WO 01/52825, WO 9310127, WO 9925719, WO 9938501, WO 9946272, WO 9967278 and WO 9967279.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33 and compounds of claims 3 to 5, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320 and US 2003096857, WO 2004/052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables Ia to If, or compounds of claims 2 to 55, WO 02/068420 especially the compounds specifically described, such as the compounds 1 to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO 03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 1 to 54, WO 03002593 especially the compounds specifically described, such compounds table 1 or of claims 2 to 15, WO03037327 especially the compounds specifically described, such compounds of examples 1 to 209, WO 0238541, WO 0230890, U.S. application Ser. No. 09/788,173 filed Feb. 16, 2001 (attorney file LA50) especially the described examples, WO99/38501 especially the described examples, WO99/46272 especially the described examples and DE19616 486 A1 especially val-pyr, val-thiazolidide, isoleucyl-thiazolidide, isoleucyl-pyrrolidide, and fumar salts of isoleucyl-thiazolidide and isoleucyl-pyrrolidide, WO 0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60.

Further preferred DPP-IV inhibitors include the specific examples disclosed in U.S. Pat. No. 6,124,305 and U.S. Pat. No. 6,107,317, International Patent Applications, Publication Numbers WO 9819998, WO 95153 09 and WO 9818763; such as 1[2-[(5 eyanopyridin-2-yl)aminoethylamino]acetyl-2-cyano-(S)-pyrrolidine and (2S)—I-[(2S)-2 amino-3,3-dimethylbutanoyl]-2-pyrrolidinecarbonitrile.

WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine. Preferred compounds described in WO03/002553 are listed on pages 9 to 11 and are incorporated into the present application by reference. Published patent application WO 0034241 and published U.S. Pat. No. 6,110,949 disclose N-substituted adamantyl-amino-acetyl-2-cyano pyrrolidines and N-(substituted glycyl)-4-cyano pyrrolidines respectively. DPP-IV inhibitors of interest are specially those cited in claims 1 to 4. In particular these applications describe the compound 1-[[(3-Hydroxy-1-adamantyl)amino]acetyl]-2-cyano-(S)-pyrrolidine (also known as LAF237).

WO 9515309 discloses amino acid 2-cyanopyrrolidine amides as inhibitors of DPP-IV and WO 9529691 discloses peptidyl derivates of diesters of alpha-aminoalkylphosphonic acids, particularly those with proline or related structures. DPP-IV inhibitors of interest are specially those cited in Table 1 to 8. In WO 01/72290 DPP-IV inhibitors of interest are specially those cited in example 1 and claims 1, 4, and 6. WO 9310127 discloses proline boronic esters useful as DPP-IV inhibitors. DPP-IV inhibitors of interest are specially those cited in examples 1 to 19. Published patent application WO 9925719 discloses sulphostin, a DPP-IV inhibitor prepared by culturing a Streptomyces microorganism. WO 9938501 discloses N-substituted 4- to 8-membered heterocyclic rings. DPP-IV inhibitors of interest are specially those cited in claims 15 to 20.

WO 9946272 discloses phosphoric compounds as inhibitors of DPP-IV. DPP-IV inhibitors of interest are specially those cited in claims 1 to 23.

Other preferred DPP-IV inhibitors are the compounds of formula I, II or III disclosed in the patent application WO 03/057200 on page 14 to 27. Most preferred DPP-IV inhibitors are the compounds specifically described on pages 28 and 29.

Published patent applications WO 9967278 and WO 9967279 disclose DPP-IV prodrugs and inhibitors of the form A-B-C where C is either a stable or unstable inhibitor of DPP-IV.

Preferably, the N-peptidyl-O-aroyl hydroxylamine is a compound of formula VII

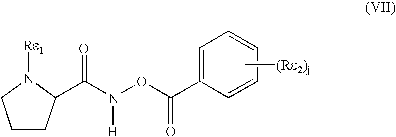

wherein
j is 0, 1 or 2;
Rε₁ represents the side chain of a natural amino acid; and
Rε₂ represents lower alkoxy, lower alkyl, halogen or nitro;
or a pharmaceutically acceptable salt thereof.

In a very preferred embodiment of the invention, the N-peptidyl-O-aroyl hydroxylamine is a compound of formula VIIIa

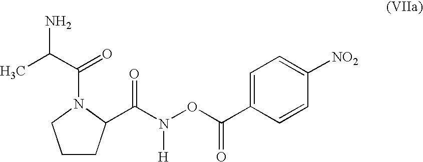

or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Most preferably the inhibitors are N-(substituted glycyl)-2-cyanopyrrolidines of formula (I)

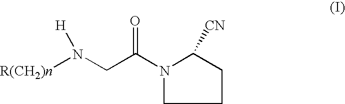

wherein

R is substituted adamantyl; and

n is 0 to 3; in free form or in acid addition salt form.

The term “substituted adamantly” refers to adamantyl, i.e., 1- or 2-adamantyl, substituted by one or more, e.g., two substituents selected from alkyl, —OR₁ or —NR₂R₃, where R₁, R₂ and R₃ are independently hydrogen, alkyl, (C₁-C₈alkanoyl), carbamyl, or —CO—NR₄R₅, where R₄ and R₅ are independently alkyl, unsubstituted or substituted aryl and where one of R₄ and R₅ additionally is hydrogen or R₄ and R₅ together represent C₂-C₇alkylene.

The term “aryl” preferably represents phenyl. Substituted phenyl preferably is phenyl substituted by one or more, e.g., two, substitutents selected from, e.g., alkyl, alkoxy, halogen and trifluoromethyl.

The term “alkoxy” refers to alkyl-O—.

The term “halogen” or “halo” refers to fluorine, chlorine, bromine and iodine.

The term “alkylene” refers to a straight chain bridge of 2 to 7 carbon atoms, preferably of 3 to 6 carbon atoms, most preferably 5 carbon atoms.

A preferred group of compounds of the invention is the compounds of formula (I), wherein the substituent on the adamantyl is bonded on a bridgehead or a methylene adjacent to a bridgehead. Compounds of formula (I), wherein the glycyl-2-cyanopyrrolidine moiety is bonded to a bridgehead, the R′ substituent on the adamantyl is preferably 3-hydroxy. Compounds of formula (I), wherein the glycyl-2-cyanopyrrolidine moiety is bonded at a methylene adjacent to a bridgehead, the R′ substituent on the adamantyl is preferably 5-hydroxy.

The present invention especially relates to a compound of formula (IA) or (IB)

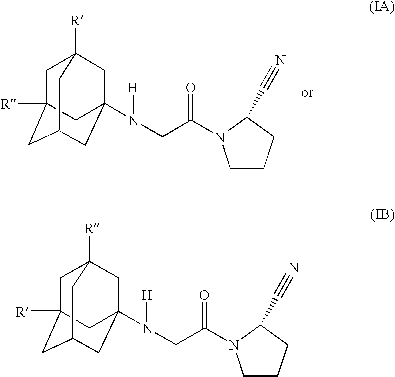

wherein
R′ represents hydroxy, C₁-C₇alkoxy, C₁-C₈alkanoyloxy or R₅R₄N—CO—O—, where R₄ and R₅ independently are C₁-C₇alkyl or phenyl which is unsubstituted or substituted by a substitutent selected from C₁-C₇alkyl, C₁-C₇alkoxy, halogen and trifluoromethyl and where R₄ additionally is hydrogen; or R₄ and R₅ together represent C₃-C₆alkylene; and
R″ represents hydrogen; or
R′ and R″ independently represent C₁-C₇alkyl;
   in free form or in form of a pharmaceutically acceptable acid addition salt.

These DPP-IV inhibitor compounds of formula (I), (IA) or (IB) are known and described in U.S. Pat. No. 6,166,063, issued Dec. 26, 2000 and WO 01/52825. Specially disclosed is (S)-1-{2-[5-cyanopyridin-2-yl)amino]ethyl-aminoacetyl)-2-cyano-pyrrolidine or (S)-1-[(3-hydroxy-1 adamantyl)amino]acetyl-2-cyano-pyrrolidine (LAF237). They can exist in free form or in acid addition salt form. Pharmaceutically acceptable, i.e., non-toxic and physiologically acceptable, salts are preferred, although other salts are also useful, e.g., in isolating or purifying the compounds of this invention. Although the preferred acid addition salts are the hydrochlorides, salts of methanesulfonic, sulfuric, phosphoric, citric, lactic and acetic acid may also be utilized.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

FE-999011 is described in the patent application WO 95/15309 page 14, as compound No. 18.

Another preferred inhibitor is the compound BMS-477118 disclosed in U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (1S,3S,5S)-2-[(2S)-2-amino-2-(3-hydroxy-tricyclo[3.3.1.1^{3,7}]dec-1-yl)-1-oxoethyl]-2-azabicyclo-[3.1.0]hexane-3-carbonitrile (M′) and its monohydrate (M″) as depicted in Formula M of the patent application WO 2004/052850 on page 3. The compound BMS-477118 is also known as saxagliptin.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound 1 and the compounds listed in the tables 1 and 2. The preferred compound is the compound 21e (table 1) of formula

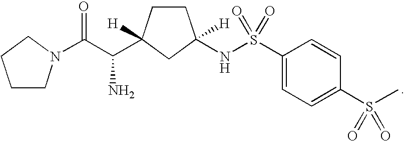

P32/98 or P3298 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below

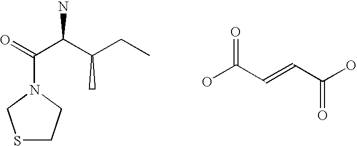

and is described in WO 99/61431 and also in Diabetes 1998, 47, 1253-1258, in the name of Probiodrug and also the compound P 93/01 described by the same company.

Other preferred DPP-IV inhibitors are the compounds disclosed in the patent application WO 02/083128 such as in the claims 1 to 5. Most preferred DPP-IV inhibitors are the compounds specifically described by the examples 1 to 13 and the claims 6 to 10.

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula

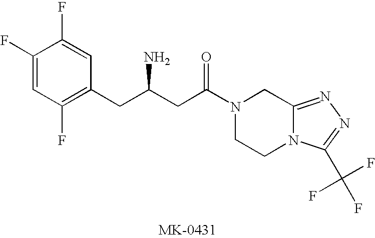

described by the example 7 and also known as MK-0431 or Sitagliptin.

Preferred DPP-IV inhibitors are also described in the patent application WO 2004/037181 especially examples 1 to 33, most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Especially preferred are 1-{2-[(5-cyanopyridin-2-yl)amino]ethylamino}acetyl-2(S)-cyano-pyrrolidine dihydrochloride (DPP728), of formula

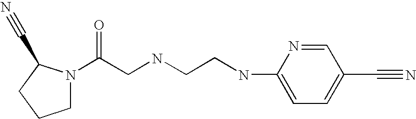

especially the dihydrochloride thereof,
and (S)-1-[(3-hydroxy-1-adamantyl)amino]acetyl-2-cyano-pyrrolidine (LAF237) of formula

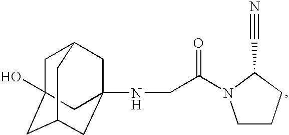

and L-threo-isoleucyl thiazolidine (compound code according to Probiodrug: P32/98 as described above), MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

DPP728 and LAF237 are the very preferred compounds and are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241. The preferred formulations for the administration of LAF237 are described in the U.S. provisional application No. 60/604,274.

Especially preferred are orally active DPP-IV inhibitors.

In each case in particular in the compound claims and the final products of the working examples, the subject matter of the final products, the pharmaceutical preparations and the claims are hereby incorporated into the present application by reference to the herein mentioned publications or patent applications.

The DPP-IV inhibitor compounds e.g. those of formula (I), and their corresponding pharmaceutically acceptable acid addition salts, may be combined with one or more pharmaceutically acceptable carriers and, optionally, one or more other conventional pharmaceutical adjuvants and administered enterally, e.g., orally, in the form of tablets, capsules, caplets, etc. or parenterally, e.g., intravenously, in the form of sterile injectable solutions or suspensions. The enteral and parenteral compositions may be prepared by conventional means.

The DPP-IV inhibitor compounds e.g. those of formula (I), and their corresponding pharmaceutically acceptable acid addition salts, may be formulated into enteral and parenteral pharmaceutical compositions containing an amount of the active substance that is effective for treating conditions mediated by DPP-IV inhibition, such compositions in unit dosage form and such compositions comprising a pharmaceutically acceptable carrier.

The DPP-IV inhibitor compounds e.g. those of formula (I), including those of each of the sub-scopes thereof and each of the examples, may be administered in enantiomerically pure form, e.g., >98%, preferably >99%; or together with the R enantiomer, e.g., in racemic form. The above dosage ranges are based on the compounds of formula (I), excluding the amount of the R enantiomer.

In view of their ability to inhibit DPP-IV, the DPP-IV inhibitor compounds e.g. those of formula (I), and their corresponding pharmaceutically acceptable acid addition salts, are useful in treating conditions mediated by DPP-IV inhibition. Based on the above and findings in the literature, it is expected that the compounds disclosed herein are useful in the treatment of conditions, such as non-insulin-dependent diabetes mellitus, arthritis, obesity, allograft transplantation and calcitonin-osteoporosis. In addition, based on the roles of glucagon-like peptides, such as GLP-1 and GLP-2, and their association with DPP-IV inhibition, it is expected that the compounds disclosed herein are useful for example, to produce a sedative or anxiolytic effect, or to attenuate post-surgical catabolic changes and hormonal responses to stress, or to reduce mortality and morbidity after myocardial infarction, or in the treatment of conditions related to the above effects which may be mediated by GLP-1 and/or GLP-2 levels.

More specifically, e.g., the DPP-IV inhibitor compounds e.g. those of formula (I), and their corresponding pharmaceutically acceptable acid addition salts, improve early insulin response to an oral glucose challenge and, therefore, are useful in treating non-insulin-dependent diabetes mellitus.

The DPP-IV inhibitor compounds especially compounds of formula I, IA or IB, useful in this invention are hygroscopic, presents stability problems, and are not inherently compatible. Consequently, there is a need to provide a free-flowing, and cohesive composition capable of being compressed into strong tablets with an acceptable in vitro dissolution profile and good stability of the active ingredients. Tablets may be defined as solid dosage pharmaceutical forms containing drug substances with or without suitable fillers. They are produced by compression or compaction of a formulation containing the active ingredient and certain excipients selected to aid in the processing and to improve the properties of the product. Tablets may be coated or uncoated and are made from powdered, crystalline materials. They may include various diluents, binders, disintegrants, lubricants, glidants and in many cases, colorants. Excipients used are classified according to the function they perform. For example, a glidant may be used to improve the flow of powder blend in the hopper and into the tablet die.

There has been widespread use of tablets since the latter part of the 19^{th} century and the majority of pharmaceutical dosage forms are marketed as tablets. Major reasons of tablet popularity as a dosage form are simplicity, low cost and the speed of production. Other reasons include stability of drug product, convenience in packaging, shipping and dispensing. To the patient or consumer, tablets offer convenience of administration, ease of accurate dosage, compactness, portability, blandness of taste, ease of administration and elegant distinctive appearance.

Tablets may be plain, film or sugar coated bisected, embossed, layered or sustained-release. They can be made in a variety of sizes, shapes and colors. Tablets may be swallowed, chewed or dissolved in the buccal cavity or beneath the tongue. They may be dissolved in water for local or topical application. Sterile tablets are normally used for parenteral solutions and for implantation beneath the skin.

In addition to the active or therapeutic ingredients, tablets may contain a number of inert materials known as excipients. They may be classified according to the role they play in the final tablet. The primary composition includes a filler, binder, lubricant and glidant. Other excipients which give physical characteristics to the finished tablet are coloring agents, and flavors in the case of chewable tablets. Without excipients most drugs and pharmaceutical ingredients cannot be directly-compressed into tablets. This is primarily due to the poor flow and cohesive properties of most drugs. Typically, excipients are added to a formulation to impart good flow and compression characteristics to the material being compressed. Such properties are imparted through pretreatment steps, such as wet granulation, slugging, spray drying spheronization or crystallization.

Lubricants are typically added to prevent the tableting materials from sticking to punches, minimize friction during tablet compression, and allow for removal of the compressed tablet from the die. Such lubricants are commonly included in the final tablet mix in amounts usually of about 1% by weight.

Other desirable characteristics of excipients include the following:
High-compressibility to allow strong tablets to be made at low compression forces;
Impart cohesive qualities to the powdered material;
Acceptable rate of disintegration
Good flow properties that can improve the flow of other excipients in the formula; and
Cohesiveness (to prevent tablet from crumbling during processing, shipping and handling).

There are three commercially important processes for making compressed tablets: wet granulation, direct compression and dry granulation (slugging or roller compaction). The method of preparation and type of excipients are selected to give the tablet formulation the desired physical characteristics that allow for the rapid compression of the tablets. After compression, the tablets must have a number of additional attributes, such as appearance, hardness, disintegrating ability and an acceptable dissolution profile. Choice of fillers and other excipients will depend on the chemical and physical properties of the drug, behavior of the mixture during processing and the properties of the final tablets. Preformulation studies are done to determine the chemical and physical compatibility of the active component with proposed excipients.

The properties of the drug, its dosage forms and the economics of the operation will determine selection of the best process for tableting. Generally, both wet granulation and direct compression are used in developing a tablet.

The dry granulation method may be used where one of the constituents, either the drug or the diluent, has sufficient cohesive properties to be tabletted. The method consists of blending, slugging the ingredients, dry screening, lubrication and compression.

The wet granulation method is used to convert a powder mixture into granules having suitable flow and cohesive properties for tableting. The procedure consists of mixing the powders in a suitable blender followed by adding the granulating solution under shear to the mixed powders to obtain a granulation. The damp mass is then screened through a suitable screen and dried by tray drying or fluidized bed drying. Alternately, the wet mass may be dried and passed through a mill. The overall process includes weighing, dry powder blending, wet granulating, drying, milling, blending lubrication and compression.

In general, powders do not have sufficient adhesive or cohesive properties to form hard, strong granules. A binder is usually required to bond the powder particles together due to the poor cohesive properties of most powders. Heat and moisture sensitive drugs cannot usually be manufactured using wet granulation. The large number of processing steps and processing time are problems due to high level manufacturing costs. Wet granulation has also been known to reduce the compressibility of some pharmaceutical excipients, such as microcrystalline cellulose.

Direct compression is regarded as a relatively quick process where the powdered materials are compressed directly without changing the physical and chemical properties of the drug. The active ingredient(s), direct compression excipients and other auxiliary substances, such as a glidant and lubricant are blended in a twin shell blender or similar low shear apparatus before being compressed into tablets. This type of mixing was believed to be essential in order to prepare “pharmaceutically acceptable” dosage forms. Some pharmaceutical scientists believe that the manner in which a lubricant is added to a formulation must be carefully controlled. Accordingly, lubricants are usually added to a granulation by gentle mixing. It is also believed that prolonged blending of a lubricant with a granulation can materially affect hardness and disintegration time for the resulting tablets. Excessive blending of lubricants with the granulate ingredients can cause water proofing of the granule and reduces tablet hardness or strength of the compressed tablet. For these reasons, high-shear mixing conditions have not been used to prepare direct compression dosage forms.

The advantages of direct compression include uniformity of blend, few manufacturing steps involved, i.e., the overall process involves weighing of powders, blending and compression, hence less cost; elimination of heat and moisture, prime particle dissociation and physical stability.

Pharmaceutical manufacturers would prefer to use direct compression techniques over wet or dry granulation methods because of quick processing time and cost advantages. However, direct compression is usually limited to those situations where the drug or active ingredient has physical characteristics required to form pharmaceutically acceptable tablets. However, one or more excipients must often be combined with the active ingredient before the direct-compression method can be used since many ingredients do not have the necessary properties. Since each excipient added to the formulation increases the tablet size of the final product, manufacturers are often limited to using the direct-compression method in formulations containing a low dose of the active ingredient per compressed tablet.

A solid dosage form containing a high-dose drug, i.e., the drug itself comprises a substantial portion of the total compressed tablet weight, could only be directly compressed if the drug itself has sufficient physical characteristics, e.g., cohesiveness, for the ingredients to be directly compressed and if the drug is properly formulated.

For an example, the DPP-IV inhibitor e.g. those of formula (I) is considered a high-dose drug. Most tablet formulations include a range of 70-85% by weight of DPP-IV inhibitor per tablet. This high-dose drug, combined with its rather poor physical characteristics for direct compression, has not permitted direct compression as a method to prepare the final tablet. In addition, the active ingredients have poor stability in presence of water, another factor militating against the use of the wet granulation method.

Another limitation of direct compression as a method of tablet manufacturing is the potential size of the compressed tablets. If the amount of active ingredient is high, a pharmaceutical formulator may choose to wet granulate the active ingredient with other excipients to attain an acceptable sized tablet with the desired amount of active ingredient.

The amount of filler, binder or other excipients needed in wet granulation is less than that required for direct compression since the process of wet granulation contributes toward the desired physical properties of the tablet.

Despite the advantages of the direct compression, such as reduced processing time and cost, wet granulation is widely-used in the industry to prepare solid dosage forms. Wet granulation is often preferred over direct compression because wet granulation has a greater chance of overcoming any problems associated with the physical characteristics of various ingredients in the formulation. This provides material which has the required flow and cohesive properties necessary to obtain an acceptable solid dosage form.

The popularity of wet granulation compared to direct compression is based on at least three advantages. First, wet granulation provides the material to be compressed with better wetting properties, particularly in the case of hydrophobic drug substances. The addition of hydrophilic excipients makes the surface of the hydrophobic drug more hydrophilic, reducing disintegration and dissolution problems. Second, the content uniformity of the solid dosage form is generally improved with wet granulation because all of the granules usually contain the same amount of drug. Lastly, the segregation of drug(s) from excipients is avoided.

Segregation could be a potential problem with direct compression. The size and shape of particles comprising the granulate to be compressed are optimized through the wet granulation process. This is because when a dry solid is wet granulated the binder “glues” particles together, so that they agglomerate into spherical granules.

As there is an important amount of metformin present in the formulation of the invention, the size and shape of the resulting tablet is problematic for an easy oral administration to a patient, as well as for an easy tablet manufacturing process which meets all the herein described requirements. Thus there is a need in the industry for techniques and pharmaceutical formulations which will allow manufacturers to prepare high-dose DPP-IV inhibitor and metformin combination tablets (high drug load). The high-dose DPP-IV inhibitor and metformin tablets have to meet all the herein listed requirements with preferably a limited number and amount of pharmaceutical excipients to reduce the size of the tablet.

It is an object of the invention to provide a formulation comprising a DPP-IV inhibitor and metformin in the form of a free-flowing, cohesive tableting powder, capable of being easily granulated or compressed into a tablet.

It is a further object of the invention to provide a high drug load tablet in unit dosage form comprising a DPP-IV inhibitor and metformin, having an acceptable dissolution profile, as well as acceptable degrees of hardness, friability and resistance to chipping, as well as a proper disintegration time and a high stability of the active ingredients in the tablet. Vildagliptin is sensitive to moisture and therefore subject to product stability issues i.e. degradation of the active ingredient. In order to overcome this problem the applicant has developed a formulation (with selected excipients) and a direct compression process (to avoid wet granulation) in order to obtain good properties tablets e.g. hardness, friability and with improved stability of the active ingredient, but with only 25% drug load.

Metformin is typically produced by a wet granulation process with a high drug load and is known to be very difficult to process. Roller compaction is also known to be unacceptable due to poor compaction properties and a direct compression process is not recommended for such high drug load formulations. Poor compressibility and tablet friability are known issues and hence were another main emphasis during the development. Other challenges identified are as follows:
Large amount of Metformin, hence large tablets and low LAF237 drug load.
Poor processing of Met.
Met is a wet granulation process and moisture is known to be detrimental to LAF.

Thus there is an unmet need to provide diabetic patients with a compressed tablet comprising between 25 and 100 mg of vilagliptin and up to 1000 mg of metformin with an acceptable tablet size, good tablet properties e.g. hardness, friability and stability of the active ingredients.

It is a further object of the invention to provide a tablet in unit dosage form comprising a DPP-IV inhibitor and metformin, having a high drug load in order to reduce the size of the tablet wherein the active ingredients remain stable.

It is a further object of the invention to provide a process for preparing a formulation or tablet comprising a DPP-IV inhibitor and metformin, or in any case a salt thereof.

The present invention provides a formulation comprising a DPP-IV inhibitor and metformin in the form of a tableting powder, capable of being compressed into a tablet having adequate size, hardness, stability, rapid disintegration time and an acceptable dissolution pattern.

In addition to the active ingredients, the tableting powder contains a number of inert materials known as excipients. They may be classified according to the role they play in the final tablet. The primary composition includes fillers, binders or diluents, lubricants, disintegrants and glidants. Other excipients which give physical characteristics to the finished tablet are coloring agents, and flavors in the case of chewable tablets. Typically, excipients are added to a formulation to impart good flow and compression characteristics to the material being compressed.

The preferred formulation of this invention comprises the following: the active ingredients which are the DPP-IV inhibitor compound and metformin, and a binder.

Examples of pharmaceutically acceptable binders include, but are not limited to, starches; celluloses and derivatives thereof, e.g., microcrystalline cellulose, hydroxypropyl cellulose hydroxylethyl cellulose and hydroxylpropylmethyl cellulose; sucrose; dextrose; corn syrup; polysaccharides; and gelatin. The binder, e.g., may be present in an amount from about 1% to about 40% by weight of the composition preferably 1% to 30% or 1% to 25% or 1% to 20%.

Optionally, one, two, three or more diluents can be added to the formulation of the invention. Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 15% to about 40% by weight of the composition. The preferred diluents include microcrystalline cellulose which is manufactured by the controlled hydrolysis of alpha-cellulose, obtained as a pulp from fibrous plant materials, with dilute mineral acid solutions. Following hydrolysis, the hydrocellulose is purified by filtration and the aqueous slurry is spray dried to form dry, porous particles of a broad size distribution. Suitable microcrystalline cellulose will have an average particle size of from about 20 nm to about 200 nm. Microcrystalline cellulose is available from several suppliers. Suitable microcrystalline cellulose includes Avicel PH 101, Avicel PH 102, Avicel PH 103, Avicel PH 105 and Avicel PH 200, manufactured by FMC Corporation. Particularly preferred in the practice of this invention is Avicel PH 102, which has the smallest surface area and pore structure. Preferably the microcrystalline cellulose is present in a tablet formulation in an amount of from about 25% to about 70% by weight. Another preferred range of this material is from about 30% to about 35% by weight; yet another preferred range of from about 30% to about 32% by weight. Another diluent is lactose. Preferably, the lactose is ground to have an average particle size of between about 50 μm and about 500 μm prior to formulating. The lactose is present in the tablet formulation in an amount of from about 5% to about 40% by weight, and can be from about 18% to about 35% by weight, and most preferred, can be from about 20% to about 25% by weight.

Optionally one, two, three or more disintegrants can be added to the formulation of the invention. Examples of pharmaceutically acceptable disintegrants include, but are not limited to, starches; clays; celluloses; alginates; gums; cross-linked polymers, e.g., cross-linked polyvinyl pyrrolidone, cross-linked calcium carboxymethylcellulose and cross-linked sodium carboxymethylcellulose; soy polysaccharides; and guar gum. The disintegrant, e.g., may be present in an amount from about 2% to about 20%, e.g., from about 5% to about 10%, e.g., about 7% about by weight of the composition. A disintegrant is also an optional but useful component of the tablet formulation. Disintegrants are included to ensure that the tablet has an acceptable rate of disintegration. Typical disintegrants include starch derivatives and salts of carboxymethylcellulose. Sodium starch glycolate is the preferred disintegrant for this formulation. Preferably the disintegrant is present in the tablet formulation in an amount of from about 0% to about 10% by weight, and can be from about 1% to about 4% by weight, and most preferred, can be from about 1.5% to about 2.5% by weight.

Optionally one, two, three or more lubricants can be added to the formulation of the invention. Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant, e.g., may be present in an amount from about 0.1% to about 5% by weight of the composition; whereas, the glidant, e.g., may be present in an amount from about 0.1% to about 10% by weight. Lubricants are typically added to prevent the tableting materials from sticking to punches, minimize friction during tablet compression and allow for removal of the compressed tablet from the die. Such lubricants are commonly included in the final tablet mix in amounts usually less than 1% by weight. The lubricant component may be hydrophobic or hydrophilic. Examples of such lubricants include stearic acid, talc and magnesium stearate. Magnesium stearate reduces the friction between the die wall and tablet mix during the compression and ejection of the tablets. It helps prevent adhesion of tablets to the punches and dies. Magnesium stearate also aids in the flow of the powder in the hopper and into the die. It has a particle size range of 450-550 microns and a density range of 1.00-1.80 g/mL. It is stable and does not polymerize within the tableting mix. The preferred lubricant, magnesium stearate is also employed in the formulation. Preferably, the lubricant is present in the tablet formulation in an amount of from about 0.25% to about 6%; also preferred is a level of about 0.5% to about 4% by weight; and most preferably from about 0.1% to about 2% by weight. Other possible lubricants include talc, polyethylene glycol, silica and hardened vegetable oils. In an optional embodiment of the invention, the lubricant is not present in the formulation, but is sprayed onto the dies or the punches rather than being added directly to the formulation.

Other conventional solid fillers or carriers, such as, cornstarch, calcium phosphate, calcium sulfate, calcium stearate, magnesium stearate, stearic acid, glyceryl mono- and distearate, sorbitol, mannitol, gelatin, natural or synthetic gums, such as carboxymethyl cellulose, methyl cellulose, alginate, dextran, acacia gum, karaya gum, locust bean gum, tragacanth and the like, diluents, binders, lubricants, disintegrators, coloring and flavoring agents could optionally be employed.

Additional examples of useful excipients which can optionally be added to the composition of the invention are described in the Handbook of pharmaceutical excipients, 3rd edition, Edited by A. H. Kibbe, Published by: American Pharmaceutical Association, Washington D.C., ISBN: 0-917330-96-X, or Handbook of Pharmaceutical Excipients (4^{th} edition), Edited by Raymond C Rowe—Publisher: Science and Practice which are incorporated herewith by reference.

Thus, in a first embodiment, the present invention concerns a high drug load pharmaceutical composition comprising between 50 to 98%, between 50% to 96%, between 60% to 98%, between 60% to 96% or between 70 to 98%, between 70% and 96%, between 80 to 98% or between 80 to 96% by weight on a dry weight basis of active ingredients, wherein the active ingredients consist of a DPP-IV inhibitor preferably vildagliptin and metformin, or in each case a pharmaceutically acceptable salt thereof.

In a second embodiment, the invention concerns a high drug load tablet or directly compressed tablet, comprising between 50 to 98%, between 50% to 96%, between 60% to 98%, between 60% to 96% or between 70 to 98%, between 70% and 96%, preferably between 80 to 98% or between 80 to 96% by weight on a dry weight basis of active ingredients, wherein the active ingredients consist of a DPP-IV inhibitor preferably vildagliptin and metformin, or in each case a pharmaceutically acceptable salt thereof.

A composition or tablet as described hereinabove, wherein metformin is in the form of granules.

A composition or tablet as described hereinabove, wherein metformin is in the form of granules and wherein the granules contain at least one pharmaceutically acceptable excipient.

A composition or tablet as described hereinabove, wherein metformin is in the form of granules and wherein the granules contain a binder.

A composition or tablet as described hereinabove, wherein metformin is in the form of granules comprising between 1 to 25% of a binder (1 to 25% of the weight of the granule on a dry weight basis).

A tablet as described herein, obtained by direct compression of the metformin granules with vildagliptin and optionally at least one pharmaceutically acceptable excipient.

A composition or tablet as described hereinabove, comprising between 1 to 25% of a binder preferably between 1 to 20% preferably between 1 and 12%, between 2.9 and 11% or between 6.5 and 9.5% or between 7.5 and 17.5% or between 12.5 and 17.5% by weight on a dry weight basis of a pharmaceutically acceptable binder.

A composition or tablet as described hereinabove, comprising at least one additional pharmaceutically acceptable excipient which is a lubricant, preferably between 0.1% to 5% between 0.1% to 2% or between 0.1% to 1.5% by weight of the composition or tablet, or between 0.1% to 1% by weight of the composition or tablet. A pharmaceutical composition or tablet as described hereinabove, wherein the lubricant is magnesium stearate.

A pharmaceutical composition or tablet as described herein, wherein the binder is selected from starches; celluloses and derivatives thereof, e.g., microcrystalline cellulose, hydroxypropyl cellulose, hydroxylethyl cellulose and hydroxylpropylmethyl cellulose; sucrose; dextrose; corn syrup; polysaccharides; and gelatin

A pharmaceutical composition or tablet as described herein, wherein the binder is selected from celluloses and derivatives thereof preferably a hydroxypropylcellulose (HPC).

The herein described ratios have been obtained on a dry weight basis for the DPP-IV inhibitors, metformin and excipients e.g. the binder.

A pharmaceutical composition as described herein which is in the form of a unit dosage form. The unit dosage form, is any kind of pharmaceutical dosage form such as capsules, tablets (preferably directly compressed tablets), granules, chewable tablets, etc.

In a further, embodiment, the present invention concerns a tablet or pharmaceutical composition comprising as active ingredients,
i) between 0.5 to 35% or between 1.5 to 35%, preferably between 0.5 to 20% or 1.5 to 20% of a DPP-IV inhibitor, preferably vildagliptin or a pharmaceutically acceptable salt thereof,
ii) between 65 to 98.5%, preferably between 80 to 98.5% of metformin or a pharmaceutically acceptable salt thereof,
   and wherein metformin is in the form of granules comprising between 1 to 25% of a binder (1 to 25% of the weight of the granule on a dry weight basis), or the herein described high load tablet or high drug load pharmaceutical composition comprising as active ingredients,
i) between 0.5 to 35% or between 1.5 to 35%, preferably between 0.5 to 20% or 1.5 to 20% of a DPP-IV inhibitor, preferably vildagliptin or a pharmaceutically acceptable salt thereof,
ii) between 65 to 98.5%, preferably between 80 to 98.5% of metformin or a pharmaceutically acceptable salt thereof,
   and wherein metformin is in the form of granules comprising between 1 to 25% of a binder (1 to 25% of the weight of the granule on a dry weight basis).

A tablet or pharmaceutical composition as described herein wherein the granules comprise between 1 to 20% preferably between 3 and 13%, between 4.9 and 12% or between 7.5 and 10.5% or between 7.5 and 17.5% or between 12.5 and 17.5% by weight on a dry weight basis of a pharmaceutically acceptable binder.

A tablet or pharmaceutical composition as described herein wherein the wherein the binder is selected from starches; celluloses and derivatives thereof, e.g., microcrystalline cellulose, hydroxypropyl cellulose, hydroxylethyl cellulose and hydroxylpropylmethyl cellulose; sucrose; dextrose; corn syrup; polysaccharides; and gelatin.

A tablet or pharmaceutical composition as described herein wherein the wherein the binder is selected from celluloses and derivatives thereof, preferably hydroxypropylcellulose (HPC).

The herein claimed compositions and tablets preferably contain at least one pharmaceutically acceptable excipient.

Additional conventional pharmaceutically acceptable excipients, at least one, e.g. 1, 2, 3 or 4, can optionally be added to the herein described formulations such as the conventional, binders, diluents, disintegrant, solid fillers or carriers described herein. Preferably the formulation does not contain more than 25% or 20% or preferably 17.5 or 15% or 11% by weight on a dry weight basis of a pharmaceutically acceptable excipient including the binder.

A tablet or pharmaceutical composition as described herein comprising between 1 and 12%, preferably between 2.9 and 11% or between 6.5 and 9.5% or between 7.5 and 17.5% or between 12.5 and 17.5% by weight on a dry weight basis of a pharmaceutically acceptable binder and optionally between 0.1 and 10% by weight on a dry weight basis of a further pharmaceutically acceptable excipient (one, two or more) e.g. between 0.1% to 2% by weight of the composition/tablet of a lubricant (e.g. magnesium stearate). Preferably, the granules comprise between 3 and 13%, between 4.9 and 12% or between 7.5 and 10.5%, or between 7.5 and 17.5% or between 12.5 and 17.5%, by weight on a dry weight basis of a pharmaceutically acceptable binder.

A tablet or pharmaceutical composition as described herein comprising between 50 to 98%, between 70 to 98%, or preferably between 80 to 98% or between 80 to 96% by weight on a dry weight basis of active ingredients, wherein the active ingredients preferably consist of vildagliptin and metformin, or in each case a pharmaceutically acceptable salt thereof.

A tablet or pharmaceutical composition as described herein comprising at least one additional pharmaceutically acceptable excipient.

A tablet or pharmaceutical composition as described herein, wherein the additional pharmaceutically acceptable excipient can be fillers, binders or diluents, lubricants, disintegrants and glidants. Other excipients which give physical characteristics to the finished tablet are coloring agents, and flavors in the case of chewable tablets.

A tablet or pharmaceutical composition as described herein comprising at least one additional pharmaceutically acceptable excipient which is a lubricant, preferably between 0.1% to 5% or between 0.1% to 2% by weight of the composition, most preferably between 0.5% to 1.5% by weight of the composition/tablet.

A tablet or pharmaceutical composition as described herein comprising between 0.1 to 5%, preferably between 0.1 to 2% or 0.5 to 1.5% of magnesium stearate.

A tablet or pharmaceutical composition as described herein, wherein the lubricant is magnesium stearate.

A tablet or pharmaceutical composition as described herein, wherein the metformin granules are produced by, wet or melt granulation, with the binder.

A tablet or pharmaceutical composition as described herein, wherein the metformin granules are produced by wet granulation with water or a solvent selected from an organic solvent such as ethanol, isopropanol, ethyl acetate, glycofurol, propylene glycol.

A tablet or pharmaceutical composition as described herein, wherein the metformin granules are produced by melt granulation. Melt granulation processes are described in many publications such as “Hot-melt extrusion Technique”: A Review; Iranian Journal of Pharmaceutical Research (2004) 3: 3-16; Rina Chokshi et al. or the review article from Jorg Breitenbach “Melt extrusion: from process to drug delivery technology”: European Journal of Pharmaceutics and Biopharmaceutics 54 (2002) 107-117, both incorporated herewith by reference.

A tablet or pharmaceutical composition as described herein, wherein vildagliptin is present in the form of drug substance.

A tablet or formulation as described herein, wherein the DPP-IV inhibitor, preferably vildagliptin, represent between 0.5 to 35% or between 1.5 to 35% of the active ingredients i.e. from DPP-IV inhibitor+metformin

A tablet or pharmaceutical composition as described herein, wherein vildagliptin is in the form of particles and wherein at least 40%, preferably 60%, most preferably 80% even more preferably 90% of vildagliptin has a particle size distribution of less than 250 μm or preferably between 10 to 250 μm or wherein at least 25% or at least 35% of the particle size distribution is between 50 to 150 μm.

A tablet or pharmaceutical composition as described herein wherein vildagliptin is in the form of particles.

A tablet or pharmaceutical composition as described herein, wherein the vildagliptin particles are produced by solvent granulation.

A tablet or pharmaceutical composition as described herein wherein the solvent used for the granulation process is preferably selected from ethanol, isopropanol, ethyl acetate, glycofurol or propylene glycol.

A pharmaceutical composition as described herein which is contained in a capsule or is in the form of a tablet preferably a compressed tablet or a directly compressed tablet. The tablet can additionally be film coated e.g. a film coating of Opadry premix.

A pharmaceutical composition as described herein, wherein the formulation represents one of the layers of a bilayer or trilayer tablet. A preferred bilayer tablet according to the invention would contain a first layer comprising a formulation of the invention and a further metformin or glitazone (e.g. pioglitazone or rosiglitazone or in any case a pharmaceutical salt thereof) formulation as a second layer.

A formulation according to the invention comprising a further active ingredient which is a glitazone e.g. pioglitazone or rosiglitazone, or an insulin secretagogues such as the sulfonylureas, e.g., Glipizide, glyburide and Amaryl; insulinotropic sulfonylurea receptor ligands such as meglitinides, e.g., nateglinide and repaglinide. The glitazone or sulfonylureas can be comprised in the metformin granules (metformin+binder+glitazone or metformin+binder+sulfonylureas) or with the LAF237 drug substance.

A bilayer or trilayer tablet, wherein the formulations according to the invention represent one layer and a glitazone e.g. pioglitazone, rosiglitazone or sulfonylureas, is present in a second layer.

Additional conventional pharmaceutically acceptable excipients (at least one, e.g. 1, 2, 3, or 4 excipitents) can optionally be added to the herein described formulations such as the conventional, diluents, disintegrant, solid fillers or carriers described herein. Preferably the formulation does not contain more than 25%, 20%, 17.5 or 13% by weight on a dry weight basis of a pharmaceutically acceptable excipient including the binder i.e. binder present in the metformin granules.

Most preferably the pharmaceutical composition comprises between 0.1 to 5%, preferably between 0.5 to 3% or 0.5 to 1.5% of a pharmaceutically acceptable lubricant, preferably magnesium stearate.

The above described compositions can comprise one or two diluents selected from microcrystalline cellulose such as Avicel PH 102 and lactose.

In the present application the reference to a pharmaceutically acceptable disintegrant means at least one disintegrant, a mixture of e.g. 2 or 3 disintegrants is also covered.

In the present application the reference to a pharmaceutically acceptable lubricant means at least one lubricant, a mixture of e.g. 2 or 3 lubricants is also covered.

Preferred DPP-IV inhibitor is LAF237, preferred diluents are microcrystalline cellulose or lactose or preferably a combination of microcrystalline cellulose and lactose, preferred disintegrant is sodium starch glycolate, preferred binder is a cellulose type binder (celluloses and derivatives thereof) e.g. HPC, and preferred lubricant is magnesium stearate.

The above described formulations are particularly adapted for the production of pharmaceutical tablets e.g. compressed tablets or direct compressed tablets, caplets or capsules and provides the necessary physical characteristics, dissolution and drug release profiles as required by one of ordinary necessary physical skill in the art. Therefore in an additional embodiment, the present invention concerns the use of any of the above described formulations, for the manufacture of pharmaceutical tablets, caplets or capsules in particular for granulation, direct compression and dry granulation (slugging or roller compaction).

The above formulations are also particularly useful for the production of tablets especially compressed tablets or direct compressed tablets.

In particular the tablets obtained with the above described formulations, have very low friability problems, very good breaking strength, improved manufacturing robustness, optimal tablet thickness to tablet weight ratios (direct compressed tablets), less water in the formulation especially directed compressed tablet, good Dispersion Disintegration time DT according to the British Pharmacopoeia 1988, good Dispersion Quality.

This present invention involves blending, granulating and compression. The choice of grades of excipients took also into consideration particle size maintained within a range that allows homogeneity of the powder mix and content uniformity of active ingredients. It prevents segregation of powders in the hopper during compression. The advantages of using the formulation of the invention is that it impart compressibility, cohesiveness and flowability of the powder blend. In addition, the compression provides competitive unit production cost, shelf life, eliminates heat and moisture, allows for prime particle dissociation, physical stability and ensures particle size uniformity.

The described advantages of the claimed compositions are also very useful for e.g. roller compaction or wet granulation, compression and direct compression or to fill capsules.

In the development of the herein described pharmaceutical compositions, the applicant has discovered that the compressed tablets especially direct compressed tablet is particularly advantageous if;
i) the particles comprising the DPP-IV inhibitor have a particle size distribution of less than 250 μm preferably between 10 to 250 μm, and/or
ii) the water content of the tablet at less than 10% after 1 week at 25° C. and 60% room humidity (RH).

Thus in a further embodiment (a), the present invention concerns a pharmaceutical formulation or a compressed tablet as described herein, wherein the dispersion contains particles comprising DPP-IV inhibitor preferably LAF237, in free form or in acid addition salt form, and wherein at least 60%, preferably 80% and most preferably 90% of the particle size distribution in the tablet is less than 250 μm or preferably between 10 to 250 μm.

The present invention concerns a pharmaceutical formulation or a compressed tablet as described herein, wherein the dispersion contains particles comprising DPP-IV inhibitor preferably LAF237, in free form or in acid addition salt form, and wherein at least 60%, preferably 80% and most preferably 90% of the particle size distribution in the tablet is greater than 10 μm.

The term “wherein metformin is in the form of granules” means that the DPP-IV inhibitor is not present in the granules containing metformin.

The term “wherein at least 60%, preferably 80% and most preferably 90%” means at least 60%, preferably at least 80% and most preferably at least 90%.

The term “wherein at least at least 25%, preferably 35% and most preferably 45%” means at least 25%, preferably at least 35% and most preferably at least 45%.

In particular the present invention concerns a pharmaceutical formulation or a compressed tablet as described herein, wherein the dispersion contains particles comprising DPP-IV inhibitor preferably LAF237, in free form or in acid addition salt form, and wherein at least 25%, preferably 35% and most preferably 45% of the particle size distribution in the tablet is between 50 to 150 μm.

In a second embodiment, this invention concerns a pharmaceutical formulation or a compressed tablet as described herein wherein the dispersion contains particles comprising DPP-IV inhibitor preferably LAF237, in free form or in acid addition salt form, and wherein;
i) at least 60%, preferably 80% and most preferably 90% of the particle size distribution in the tablet is less than 250 μm preferably between 10 to 250 μm,
ii) the water content of the tablet is less than 10% after 1 week at 25° C. and 60% RH.

Preferably this invention concerns a pharmaceutical formulation or a compressed tablet as described herein, wherein the dispersion contains particles comprising DPP-IV inhibitor preferably LAF237, in free form or in acid addition salt form, and wherein;
i) at least 25%, preferably 35% and most preferably 45% of the particle size distribution in the tablet is between 50 to 150 μm,
ii) the water content of the tablet is less than 10% after 1 week at 25° C. and 60%

RH. pr the water content of the tablet is less than 5% after 1 week at 25° C. and 60% RH.

Preferably the DPPIV particles especially the LAF237 particles comprise more than 70% of DPPIV inhibitor, most preferably more than 90% or 95% and even more preferably more than 98% of DPPIV inhibitor.

Preferably the LAF237 particles comprise more than 70% of LAF237, most preferably more than 90% or 95% and even more preferably more than 98% of LAF237.

It has been discovered that the selected particle size distribution of DPPIV inhibitor especially LAF237 were particularly important to provide the best compaction of the tablets.

The preferred excipients with an adapted particle size distribution can be picked from e.g. Handbook of Pharmaceutical Excipients (4^{th} edition), Edited by Raymond C Rowe—Publisher: Science and Practice.

Particle size of drug, e.g. LAF237 particles size, is controlled by crystallisazion, drying and/or milling/sieving (non limiting examples are described below). Particle size can also be comminuted using roller compaction and milling/sieving. Producing the right particle size is well known and described in the art such as in “Pharmaceutical dosage forms: volume 2, 2nd edition, Ed.: H. A. Lieberman, L. Lachman, J. B. Schwartz (Chapter 3: SIZE REDUCTION)”.

Process to obtain the proper LAF237 particle size is also described in the patent application WO 2005/067976 which is incorporated herein by reference.

Multiple particle sizes have been studied and it has been discovered that the herein described specific size range provides good results for compaction.

PARTICLE SIZE DISTRIBUTION ESTIMATION BY ANALYTICAL SIEVING: Particle size distribution is measured using Sieve analysis, Photon Correlation Spectroscopy or laser diffraction (international standard ISO 13320-1), or electronic sensing zone, light obstruction, sedimentation or microscopy which are procedures well known by the person skilled in the art. Sieving is one of the oldest methods of classifying powders by particle size distribution. Such methods are well known and described in the art such as in any analytical chemistry text book or by the United State Pharmacopeia's (USP) publication USP-NF (2004—Chapter 786—(The United States Pharmacopeial Convention, Inc., Rockville, Md.)) which describes the US Food and Drug Administration (FDA) enforceable standards. The used techniques are e.g. described in Pharmaceutical dosage forms: volume 2, 2nd edition, Ed.: H. A. Lieberman, L. Lachman, J. B. Schwartz is a good example. It also mentions (page 187) additional methods: Electronic sensing zone, light obstruction, air permeation, sedimentation in gas or liquid.

In an air jet sieve measurement of particle size, air is drawn upwards, through a sieve, from a rotating slit so that material on the sieve is fluidised. At the same time a negative pressure is applied to the bottom of the sieve which removes fine particles to a collecting device. Size analyses and determination of average particle size are performed by removal of particles from the fine end of the size distribution by using single sieves consecutively. See also “Particle Size Measurement”, 5th Ed., p 178, vol. 1; T. Allen, Chapman & Hall, London, UK, 1997, for more details on this. For a person skilled in the art, the size measurement as such is thus of conventional character.

Water content of the tablet can be measured using Loss on drying method or Karl-Fischer method which are well known methods to the person skilled in the art (e.g. water content can be measured by loss on drying by thermogrametry). Such methods are well known and described in the art such as in any analytical chemistry text book (J. A. Dean, Analytical Chemistry Handbook, Section 19, McGraw-Hill, New York, 1995) or by the United State Pharmacopeia's (USP) publication USP-NF (2004) which describes the US Food and Drug Administration (FDA) enforceable standards ((2004—USP—Chapter 921).

This invention provides in particular a compressed tablet or direct compressed tablet which is capable of dispersing in water within a period of 15 to 50 minutes or 20-45 minutes to provide a dispersion which is capable of passing through a sieve screen with a mesh aperture of 710 μm in accordance with the herein defined British Pharmacopoeia test for dispersible tablets.

A tablet according to the invention, as well as being quickly dispersible in water, has the added advantage that it meets the British Pharmacopoeia (B.P.) test for dispersible tablets in respect of dispersion times and dispersion quality (i.e. passage through a 710 μm sieve).

Preferably the dispersion time of a tablet according to the invention is less than 15 minutes, more preferably less than 12 minutes and most preferably less than 10 minute.

A further advantage of the tablets according to invention is that because a relatively fine dispersion is formed the tablet will have a lower dissolution time and thus the drug may be absorbed into the blood stream much faster. Furthermore the fast dispersion times and relatively fine dispersions obtained with tablets according to the invention are also advantageous for swallowable tablets. Thus tablets according to the invention can be presented both for dispersion in water and also for directly swallowing. Those tablets according to the invention that are intended for swelling are preferably film-coated to aid swallowing.

In a further embodiment the present invention concerns a pharmaceutical formulation or a compressed tablet as described herein wherein

i) between 0 and 45 minutes 90 to 99.5% of LAF237 is released, and
ii) between 10 and 45 minutes 70 to 99% of metformin is released.

The Paddle method to measure the drug dissolution rate (% of release) is used with 1000 ml of 0.01 N HCl. Such methods are well known and described in the art such as in any analytical chemistry text book or by the United State Pharmacopeia's (USP) publication USP-NF (2004—Chapter 711) which describes the US Food and Drug Administration (FDA) enforceable standards.

The invention also provides a process for preparing a pharmaceutical formulation comprising a DPP-IV inhibitor preferably LAF237 or pharmaceutical salts thereof and metformin or pharmaceutical salts thereof, which comprises:
i) granulating metformin and a binder,
ii) drying granules containing metformin and the binder,
iii) blending the DPP-IV inhibitor, preferably LAF237, drug substance with the granules containing metformin and the binder,
iv) optionally a lubricant e.g. magnesium stearate is blended with the mixture obtained on step iii),

The invention also provides a process for preparing a pharmaceutical tablet comprising a DPP-IV inhibitor preferably LAF237 or pharmaceutical salts thereof and metformin or pharmaceutical salts thereof, which comprises;
i) granulating metformin and a binder,
ii) drying granules containing metformin and the binder,
iii) blending the DPP-IV inhibitor, preferably LAF237, drug substance with the granules containing metformin and the binder,
iv) optionally a lubricant e.g. magnesium stearate is blended with the mixture obtained on step iii),
v) compressing the resulting blend to form tablets in unit dosage form.

The resulting blend is in the form of a tableting powder, capable of being compressed into a tablet.

The final moisture level of the granulation after drying (LOD) can also be critical in obtaining adequate compaction properties and flow of the Metformin wet granulation (if LOD is to low the compaction properties and tablet friability are poor, while if the LOD is to high the granulation will cause significant picking and/or will begin to form aggregates and restrict powder flow). The proposed target LOD is ˜2% (range of 0.5 to 3.5 preferably a range of 1.5 to 2.4%).

Therefore, in a preferred embodiment during step ii) the granules are dried to an LOD of 0.5-3.5% preferably of 1.5-2.4%. (LOD: Loos On Drying (method defined in USP)

Preferably the granulation of step i) is a wet granulation or a melt granulation.

Unexpected good results have been observed if metformin and the binder are granulated by melt granulation (step i)). The obtained final formulations or tablets exhibit the herein described advantages e.g. improved hardness, low friability, good compactibility, dissolution and stability.

Thus in a preferred aspect, metformin and the binder are blended and the blend is passed through an extruder for melt granulation.

Preferably, the extruder is set at between 140 and 220° C., or between 155 an 205° C. or between 170 and 190° C. at mixing zone.

Preferably, the compression step v), is a direct compression of the blend resulting from steps iii) or iv).

In further embodiments, the above described processes can comprise:
A step i) in order that at the end of step ii) metformin is in the form of granules comprising between 1 to 25% or between 1 to 20% preferably between 1 to 20%, most preferably between 3 and 13%, between 4.9 and 12% or between 7.5 and 10.5% or between 7.5 and 17.5% or between 12.5 and 17.5% by weight on a dry weight basis of a pharmaceutically acceptable binder.
A step i) wherein at least one further pharmaceutically acceptable excipitent such as a diluent or a disintegrant is added to the mixture to be blended. Preferably the further pharmaceutically acceptable excipitent(s) do not represent more than 25% preferably less than 17.5% or 15% by weight on a dry weight basis of the granule weight.
A step iii) wherein at least one further pharmaceutically acceptable excipitent such as a diluent or a disintegrant is added to the mixture to be blended.
A further coating step is applied to the resulting compressed core (tablet).
The compressed cores are optionally dried to an LOD of <1% preferably <0.5% prior to tablet coating.

Preferred DPP-IV inhibitor is LAF237, preferred diluents are microcrystalline cellulose or lactose or preferably a combination of microcrystalline cellulose and lactose, preferred disintegrant is sodium starch glycolate, and preferred lubricant is magnesium stearate.

Before step (1) a sieving step is preferably applied to the formulation for basic delumping i.e. to get rid of any agglomerates/cakes. Before step (3) a sieving step is preferably applied to LAF237, before it is added to the metformin granules.

In an other embodiment, the present invention covers capsule comprising the above described pharmaceutical compositions.

The final product is prepared in the form of tablets, capsules or the like by employing conventional tableting or similar machinery.

A tablet obtained by one of the herein described process which has a hardness comprised between 14 kp and 30 kp at a compression force of 15 kN, and/or a friability between 0.5% and 0.18% at a compression force of 15 to 20 kN.

Most preferably the DPP-IV inhibitor for the herein described formulations, compressed tablets or processes is selected from 1-{2-[(5-cyanopyridin-2-yl)amino]ethylamino}acetyl-2(S)-cyano-pyrrolidine dihydrochloride, (S)-1-[(3-hydroxy-1-adamantyl)amino]acetyl-2-cyano-pyrrolidine, L-threo-isoleucyl thiazolidine, MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

Most preferably the DPP-IV inhibitor is 1-[3-hydroxy-adamant-1-ylamino)-acetyl]-pyrrolidine-2(S)-carbonitrile (LAF237 or vildagliptin) or a pharmaceutical salt thereof.

The dosage of (S)-1-[(3-hydroxy-1-adamantyl)amino]acetyl-2-cyano-pyrrolidine (vildagliptin) is preferably between 10 and 150 mg daily, most preferably between 25 and 150 mg or 50 and 100 mg or 25 to 100 mg daily. Preferred examples of daily oral dosage are 25, 30, 35, 45, 50, 55, 60, 70, 80, 90, or 100 mg. The application of the active ingredient may occur up to three times a day, preferably one or two times a day.

Glitazones which can be combined to the formulation of the invention in the form of a triple combination are well known in the art and described in many publications.

Glitazones under development are AZ242 (AstraZeneca) phase 2; KRP-297 (Kyorin, licensed to Merck) phase 1-2; MCC-555 (Mitsubishi Chemicals, licensed to J&J) phase 2; JTT-501 (Japan Tobacco, licensed to Pharmacia) phase 2.

The glitazones 5-{[4-(2-(5-ethyl-2-pyridyl)ethoxy)phenyl]-methyl}thiazolidine-2,4-dione (pioglitazone, EP 0 193 256 A1), 5-{[4-(2-(methyl-2-pyridinyl-amino)-ethoxy)phenyl]methyl}-thiazolidine-2,4-dione (rosiglitazone, EP 0 306 228 A1), 5-{[4-((3,4-dihydro-6-hydroxy-2,5,7,8-tetramethyl-2H-1-benzopyran-2-yl)methoxy)-phenyl]-methyl}thiazolidine-2,4-dione (troglitazone, EP 0 139 421), (S)-((3,4-dihydro-2-(phenyl-methyl)-2H-1-benzopyran-6-yl)methyl-thiazolidine-2,4-dione (englitazone, EP 0 207 605 B1), 5-(2,4-dioxothiazolidin-5-ylmethyl)-2-methoxy-N-(4-trifluoromethyl-benzyl)benzamide (KRP297, JP 10087641-A), 5-[6-(2-fluoro-benzyloxy)naphthalen-2-ylmethyl]thiazolidine-2,4-dione (MCC555, EP 0 604 983 B1), 5-{[4-(3-(5-methyl-2-phenyl-4-oxazolyl)-1-oxopropyl)-phenyl]-methyl}-thiazolidine-2,4-dione (darglitazone, EP 0 332 332), 5-(2-naphthylsulfonyl)-thiazolidine-2,4-dione (AY-31637, U.S. Pat. No. 4,997,948), 5-{[4-(1-methyl-cyclohexyl)methoxy)-phenyl]methyl}-thiazolidine-2,4-dione (ciglitazone, U.S. Pat. No. 4,287,200) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The preparation of DRF2189 and of 5-{[4-(2-(2,3-dihydroindol-1-yl)ethoxy)phenyl]methyl}-thiazolidine-2,4-dione is described in B. B. Lohray et al., J. Med. Chem. 1998, 41, 1619-1630; Examples 2d and 3g on pages 1627 and 1628. The preparation of 5-[3-(4-chlorophenyl])-2-propynyl]-5-phenylsulfonyl)-thiazolidine-2,4-dione and the other compounds in which A is phenylethynyl mentioned herein can be carried out according to the methods described in J. Wrobel et al., J. Med. Chem. 1998, 41, 1084-1091.

In particular, MCC555 can be formulated as disclosed on page 49, lines 30 to 45, of EP 0 604 983 B1; englitazone as disclosed from page 6, line 52, to page 7, line 6, or analogous to Examples 27 or 28 on page 24 of EP 0 207 605 B1; and darglitazone and 5-{4-[2-(5-methyl-2-phenyl-4-oxazolyl)-ethoxy)]benzyl}-thiazolidine-2,4-dione (BM-13.1246) can be formulated as disclosed on page 8, line 42 to line 54 of EP 0 332 332 B1. AY-31637 can be administered as disclosed in column 4, lines 32 to 51 of U.S. Pat. No. 4,997,948 and rosiglitazone as disclosed on page 9, lines 32 to 40 of EP 0 306 228 A1, the latter preferably as its maleate salt. Rosiglitazone can be administered in the form as it is marketed e.g. under the trademark AVANDIA™. Troglitazone can be administered in the form as it is marketed e.g. under the trademarks ReZulin™, PRELAY™, ROMOZIN™ (in the United Kingdom) or NOSCAL™ (in Japan). Pioglitazone can be administered as disclosed in Example 2 of EP 0 193 256 A1, preferably in the form of the monohydrochloride salt. Corresponding to the needs of the single patient it can be possible to administer pioglitazone in the form as it is marketed e.g. under the trademark ACTOS™. Ciglitazone can, for example, be formulated as disclosed in Example 13 of U.S. Pat. No. 4,287,200.

For administration of a glitazone to an adult diabetic patient (body weight: 50 kg), for instance, the dose per day is usually 0.01 to 1000 mg, preferably 0.1 to 500 mg. This dose can be administered once to several times a day. Especially, when pioglitazone hydrochloride is employed as the insulin sensitizer, the dose of pioglitazone hydrochloride per day is usually 7.5 to 60 mg, preferably 15 to 45 mg. When troglitazone is employed as the insulin sensitizer, the dose of troglitazone per day is usually 100 to 1000 mg, preferably 200 to 600 mg. When rosiglitazone (or its maleate) is employed as the insulin sensitizer, the dose of rosiglitazone per day is usually 1 to 12 mg, preferably 2 to 12 mg.

The glitazone is preferably pioglitazone, pioglitazone hydrochloride, troglitazone or rosiglitazone (or its maleate salt), especially preferably pioglitazone hydrochloride.

The dose of ACTOS® (pioglitazone) should not exceed 45 mg once daily in monotherapy or in combination with sulfonylurea, metformin, or insulin. ACTOS in combination with metformin may be initiated at 15 mg or 30 mg once daily. The current metformin dose can be continued upon initiation of ACTOS therapy. It is unlikely that the dose of metformin will require adjustment due to hypoglycemia during combination therapy with ACTOS. ACTOS is available in 15 mg, 30 mg, and 45 mg tablets AVANDIA® (rosiglitazone) may be administered either at a starting dose of 4 mg as a single daily dose or divided and administered in the morning and evening. For patients who respond inadequately following 8 to 12 weeks of treatment, as determined by reduction in FPG, the dose may be increased to 8 mg daily as monotherapy or in combination with metformin. The dose of AVANDIA should not exceed 8 mg daily, as a single dose or divided twice daily. AVANDIA is available in 2 mg, 4 mg, and 8 mg tablets

The dosage of antidiabetic therapy with metformin should be individualized on the basis of effectiveness and tolerability while not exceeding the maximum recommended daily dose of metformin which is 2,000 mg. Metformin has been widely prescribed for lowering blood glucose in patients with NIDDM and is marketed in 500, 750, 850 and 1000 mg strengths. However, because it is a short acting drug, metformin requires twice-daily or three-times-daily dosing (500-850 mg tab 2-3/day or 1000 mg bid with meals). Preferably the dosage used in the present invention is between 250 and 2000 mg preferably between 250 and 1000 mg. A pharmaceutical composition, tablet or capsule according to the herein described invention, comprising 250 mg, 500 mg, 850 mg or 1000 mg of metformin or a pharmaceutical salt thereof.

Thus in a further embodiment, the present invention concerns a tablet or formulation of the invention, wherein the active ingredients consist of;
i) 50 to 2000 mg of metformin, preferably 250 to 1000 mg of metformin
ii) 25 to 100 mg of a DPP-4 inhibitor preferably vildagliptin.

The present invention also concerns a pharmaceutical unit dosage form, preferably a tablet or capsule, comprising a formulation of the invention, and wherein the active ingredients consist of;
i) 50 to 2000 mg of metformin, preferably 250 to 1000 mg of metformin
ii) 25 to 100 mg of a DPP-4 inhibitor preferably vildagliptin preferably 25 to 50 mg of vildagliptin.

The present invention also concerns a pharmaceutical unit dosage form, preferably a tablet or capsule, comprising a formulation of the invention, and wherein the active ingredients consist of;
i) 25 mg of vildagliptin and 250 mg of metformin, or in any case a pharmaceutical salt thereof,
ii) 25 mg of vildagliptin and 500 mg of metformin, or in any case a pharmaceutical salt thereof,
iii) 25 mg of vildagliptin and 850 mg of metformin, or in any case a pharmaceutical salt thereof,
iv) 25 mg of vildagliptin and 1000 mg of metformin, or in any case a pharmaceutical salt thereof,
v) 50 mg of vildagliptin and 500 mg of metformin, or in any case a pharmaceutical salt thereof,
vi) 50 mg of vildagliptin and 850 mg of metformin, or in any case a pharmaceutical salt thereof, or
vii) 50 mg of vildagliptin and 1000 mg of metformin, or in any case a pharmaceutical salt thereof.

The present invention also concerns a formulation or tablet of the invention, wherein;

a) the active ingredients consist of;
i) 25 mg of vildagliptin and 250 mg of metformin, or in any case a pharmaceutical salt thereof,
ii) 25 mg of vildagliptin and 500 mg of metformin, or in any case a pharmaceutical salt thereof,
iii) 25 mg of vildagliptin and 850 mg of metformin, or in any case a pharmaceutical salt thereof,
iv) 25 mg of vildagliptin and 1000 mg of metformin, or in any case a pharmaceutical salt thereof,
v) 50 mg of vildagliptin and 500 mg of metformin, or in any case a pharmaceutical salt thereof,

vi) 50 mg of vildagliptin and 850 mg of metformin, or in any case a pharmaceutical salt thereof, or
vii) 50 mg of vildagliptin and 1000 mg of metformin, or in any case a pharmaceutical salt thereof,
   and
b) metformin is in the form of granules comprising between 1 to 25% of a binder (1 to 25% of the weight of the granule on a dry weight basis), between 1 to 20% of a binder, or between 7.5 and 17.5% of a binder,
c) the composition or tablet comprises, between 50 to 98%, between 50% to 96%, between 60% to 98%, between 60% to 96% or between 70 to 98%, between 70% and 96%, between 80 to 98% or between 80 to 96% by weight on a dry weight basis of active ingredients,
d) the composition optionally comprises at least one additional excipient such as between 0.1% and 2% magnesium state.

The present invention also concerns a pharmaceutical unit dosage form, preferably a tablet or capsule, comprising a formulation of the invention, and wherein the active ingredients consist of;
i) between 50 to 2000 mg of metformin, preferably between 250 to 1000 mg of metformin,
ii) between 25 to 100 mg of a DPP-4 inhibitor preferably vildagliptin preferably between 25 to 50 mg of vildagliptin, and
iii) between 2 to 50 mg of a glitazone, preferably between 2 to 8 mg of rosiglitazone or 15 to 45 mg of pioglitazone

Thus in a further embodiment, the present invention concerns a tablet of the invention, wherein;
the tablet hardness is comprised between 60 and 340 N,
the tablet friability is lower than 0.8%, and
the tablet thickness is comprised between 4.5 and 8.3 mm.

Thus in a further embodiment, the present invention concerns a tablet of the invention, wherein;
the tablet hardness is comprised between 60 and 340 N,
the tablet friability is lower than 0.8%,
the tablet thickness is comprised between 4.5 and 8.3 mm, and
at lest 70% of vildagliptin is dissolved within 30 minutes,
at least 80% of metformin HCl is dissolved within 45 minutes,
   by using the Paddle method.

In a further embodiment, the present invention concerns a tablet or formulation of the invention, wherein metformin is in the form of its HCl salt.

Any of the herein described compositions or tablet, comprising between 50 to 98%, between 50% to 96%, between 60% to 98%, between 60% to 96%, or between 70 to 98%, between 70% and 96%, or between 80 to 98% or 80 to 96% by weight on a dry weight basis of active ingredients, wherein the active ingredients consist of vildagliptin and metformin, or in each case a pharmaceutically acceptable salt thereof.

In a further aspect, the present invention concerns the use of the herein described formulations, capsules, tablets, compressed tables, direct compressed tablets for the treatment of conditions, such as non-insulin-dependent diabetes mellitus, arthritis, obesity, allograft transplantation, calcitonin-osteoporosis, Heart Failure, Impaired Glucose Metabolism), IGT (Impaired Glucose Tolerance), neurodegenerative diseases such as Alzheimer's and Parkinson disease, modulating hyperlipidemia, modulating conditions associated with hyperlipidemia or for lowering VLDL, LDL and Lp(a) levels, cardiovascular or renal diseases e.g. diabetic cardiomyopathy, left or right ventricular hypertrophy, hypertrophic medial thickening in arteries and/or in large vessels, mesenteric vasculature hypertrophy, mesanglial hypertrophy, neurodegenerative disorders and cognitive disorders, to produce a sedative or anxiolytic effect, to attenuate post-surgical catabolic changes and hormonal responses to stress, to reduce mortality and morbidity after myocardial infarction, the treatment of conditions related to the above effects which may be mediated by GLP-1 and/or GLP-2 levels.

In each case in particular in the compound claims, the final products of the working examples, the subject matter of the final products, the analytical and measurement methods (e.g. USP documents) the methods to obtain the right particles size, the pharmaceutical preparations, the excipients and the claims are hereby incorporated into the present application by reference to the herein mentioned publications or patent applications. This invention is further illustrated by the following examples:

### EXAMPLE 1

### Manufacturing Process

Due to the Metformin drug substance hardening during storage, a decompaction process using an oscillating mill (Frewitt) fitted with a 1.68 mm screen is required. The Metformin is then premixed with HPC-EXF (EXF: Manufacturer's (Aqualon's) grade designation for viscosity and particle size, x=xtrafine. HF=no meaning but viscosity designation that can be compared to other HPC grades, HF, GF, LF, EF) for 1-2 minutes in a high shear mixer. A 9% HPC solution (w/w) is pumped into the high shear granulator at a fixed rate (4 minutes) until adequate granules are formed (total amount of water ˜7%). The granulation is then dried in a fluid bed dryer to a final LOD (loss on drying) of ˜2% (range 1.5 to 2.4%). The dried granulation is passed through either a Fitzmill (fitted with a 0.078″ or 2 mm screen) or a Frewitt oscillator (fitted with a 1.68 mm screen). The LAF237 drug substance is passed through a 1 mm hand screen and blended with the milled Metformin granulation for 300 rotations in a bin blender. The magnesium stearate is also passed through a 1 mm hand screen and blended with the Met/LAF mixture for 60 rotations. The blend is then compressed on a rotary tablet press. The compressed cores are dried to an LOD of <0.5% prior to tablet coating. Approximately a 5 mg/cm² coating weight is applied during the coating process.

Process parameters used to manufacture batches of the herein described formulations comprising Metformin:LAF237 core batches at 5:1, 10:1, 20:1 and 40:1 ratios

| Manufacturing | | |
|---|---|---|
| process steps | Process parameter | Set point (range) |
| Pre Mixing | Time | 2 minutes |
| Milling | Mesh size | 1.68 or 2.0 mm |
| Granulation + | Amount of water | 7% of granulation amount |
| (metformin + | | |
| binder) | | |
| | Rate of addition | 4 minutes (~200 ml/min) |
| | Kneading time | 2 minutes after water |
| | | addition |
| | Plough/chopper speed | Low (setting 1) |
| Mixing (LAF237 + | Time (number of | 15 minutes (300 rotations) |
| (metformin + | rotations) | |
| binder) granules | | |
| Sieving | Mesh size | 1 mm |
| Final mixing | Time (number of | 3 minutes (60 rotations) |
| (final blend, | rotations) | |
| including e.g. | | |
| optional lubricant) | | |
| Compression | Compression speed | 40 rpm |
| | Compression force | 10-23 kN |

Description of manufacturing equipment used for the herein described formulation development

| Equipment | Size/model | Unit operation |
|---|---|---|
| Oscillator | Frewitt | Screening/ |
| | | decompaction |
| High shear mixer | 25 liter Collette Gral | Granulating |
| Convection dryer | GPCG5 Fluid bed | Drying |
| Hammer conventional mill | Fitzmill | Screening |
| Bin or container mixer | 10 and 25 liter container | Blending |
| Tablet press | Manesty Beta | Tabletting |
| Coating pan perforated | Compulab | Coating |

### Batch Sizes Tested

The batch size for the exploratory batches were typically <1.0 kg. During formulation development, the wet granulation was completed in a 25 L Collette Gral mixer with batch sizes ranging from 3.0 to 6.0 kg.

### Statement on the Up-Scaling Potential and Robustness of the Final Process

All process incorporated with the manufacture of the Metformin wet granulation and drying processes as well as the mixing, compression and coating are standard processes and use standard equipment. The FBD (fluid bed dryer) drying process end-point (1.5-2.4%) LOD.

Since the moisture level of the dried granulation could have significant impact on tabletting properties, all granulations are preferably prepared using a KG5 mixer and dried in an oven to an LOD of approximately 2% (preferred range 1.5-2.4%).

### Manufacturing Process: Alternative

Step 1: Sieve the Metformin and HPC through a 1700 μm screen. Place sieved ingredients into a diffusion blender and preblend at 20 rpm for 200 rotations.
Step 2: Pass the blend through a twin screw extruder set at 180° C. (at mixing zone)—Melt granulation.
Step 3: Sieve the granulation through a 500 μm screen using a frewitt (milling step).
Step 4: Sieve LAF237 through a 500 μm screen and blend with granulation of step 3, at 20 rpm for 300 rotations.
Step 5: Sieve magnesium stearate through a 1000 μm screen and blend at 20 rpm for 60 rotations.
Step 6: Compression of the resulting composition
Step 7: Film coating

### EXAMPLE 1B

### Preparation of Metformin Granules Using the Melt Granulation Process

| | | |
|---|---|---|
| Ingredient | Percentage (w/w) | Amount per tablet (mg) |

Internal phase

| | |
|---|---|
| metformin HCl | 1000 |
| hydroxypropyl cellulose | 99 |

External phase

| | |
|---|---|
| magnesium stearate | 11 |
| vildagliptin | 50 |
| Total | 1160 |

The internal phase ingredients i.e. metformin hydrochloride, and hydroxypropyl cellulose available as KLUCEL EXF from Hercules Chemical Co. (Wilmington, Del.) are combined and blended in a bin blender for about two hundred rotations. The blend is introduced into the feed section, or hopper, of a twin screw extruder. A suitable twin screw extruder is the PRISM 16 mm pharmaceutical twin screw extruder available from Thermo Electron Corp. (Waltham, Mass.).

Located at the end of the twin screw extruder is a die with a bore of approximately three mm. The twin screw extruder is configured with five individual barrel zones, or sections, that can independently adjusted to different parameters. Starting from the hopper to the die, the zones are respectively heated to the following temperatures: 40° C., 110° C., 130° C., 170° C. and 185° C. The temperatures of the heating zones do not exceed the melting temperature of metformin hydrochloride which is approximately 232° C. The screw speed is set to 150 rpm, but can be as high as 400 rpm, and the volumetric feed rate is adjusted to deliver between about 30 to 45 grams of material per minute. The throughput rate can be adjusted from 4 g/min to 80 g/min.

The extrudate, or granules, from the extruder are then cooled to room temperature by allowing them to stand from approximately fifteen to twenty minutes. The cooled granules, are subsequently sieved through a 500 micrometer screen (i.e., a one mm screen).

For the external phase, the magnesium stearate is sieved through a 1000 micrometer screen and vildagliptin drug substance is first passed through a 500 micrometer screen. Vildagliptin is then blended with the obtained granules using a suitable bin blender for approximately 150 or 300 rotations. The magnesium stearate is blended with the resulting mixture for 50 or 70 rotations. The resulting final blend is compressed into tablets using a conventional rotary tablet press (Manesty Beta Press) using a compression force ranging between 6 kN and 25 kN. The resulting tablets are monolithic and having a hardness ranging from 5 kP to 35 kP. Tablets having hardness ranging from 15 kP to 35 kP resulted in acceptable friability of less than 1.0% w/w after five hundred drops. Moreover, these tablets have a disintegration time of less than equal to twenty minutes with discs at 37° C. in 0.1 N HCl.

### EXAMPLE 2

### A. Summary of Extended Compatibility Tests

Excipient compatibility study of the herein described formulations with standard excipients at 50° C./75% (open) for 4 weeks was conducted. Based on the compatibility results, the data indicate that the herein described formulations and tablets provided less degradation of metformin or LAF237.

### B. Stability Protocol

Stability studies at 25° C./60% RH, 30/65% RH and 40° C./75% RH was conducted in induction sealed HDPE (high density polyethylene) bottles with desiccant and at 40° C./75% RH open without desiccant (Open). Stability conditions at different time points have shown better result with the herein described formulations and tablets.

RH=relative humidity

TABLE i
Exploratory formulation stability storage conditions.

| | Storage conditions | | | |
|---|---|---|---|---|
| | | 30° C./ | 40° C./ | 40° C./ |
| Interval | 25° C./60% RH | 65% RH | 75% RH | 75% RH, Open |
| 3 W | | | | X |
| 6 W | | | X | X |
| 3 M | X | | X | |
| 6 M | X | | | |

TABLE ii
Melt granulation and low moisture series stability storage conditions

| | Storage conditions | | | |
|---|---|---|---|---|
| | | 30° C./ | 40° C./ | 40° C./ |
| Interval | 25° C./60% RH | 65% RH | 75% RH | 75% RH, Open |
| 3 W | | | | X |
| 6 W | | | X | X |
| 3 M | X | [X] | X | |
| 6 M | X | [X] | | |
| 12 M | X | [X] | | |

[ ] = optional test

Stability results: Good stability have been obtained with the herein described formulations and tablets.

### Stability of Low Moisture Series Formulations, Met:LAF 40:1 Ratio

(Metformin Directly Compressed) (Pre-granulated material sold as a “new grade” for direct compression in to tablets)+LAF237 (solvent granulation) results in a LAF237 Total degradation of 2.9% in the 40° C./75% RH+6 weeks closed storage conditions.

(Metformin water granulated with 6.6% of HPC)+LAF237 (solvent granulation) (claimed formulation) results in a LAF237 Total degradation of 0.9% in the 40° C./75% RH+6 weeks closed storage conditions.

Co-granulation of (metformin+LAF237) with 6.6% HPC results in a LAF237 Total degradation of 6.6% in the 40° C./75% RH+6 weeks closed storage conditions.

Furthermore, the applicant has tested many other formulations and has discovered that a formulation, (e.g. tablet in unit dosage form), comprising a DPP-IV inhibitor and metformin, and having a high drug load provides better stability results, especially if a binder is present preferably if HPC is present.

### C. Test Conditions for Dissolution Rate

The method that was selected was based on the results from earlier method development studies showing similar release profiles of Metformin and LAF237 at different pH's (0.01 N HCl, pH 4.5 and pH 6.8 buffer) as well as from paddles or baskets (50 and 100 rpm).

### USP Apparatus: I (Baskets)

### Rotation Speed: 100 rpm

Dissolution Medium: 0.01 N HCl, degassed.

### Volume: 900 ml

The dissolution was performed (n=3) for initial samples only. Dissolution on stability samples have shown good results with the herein described formulations and tablets. The dissolution rate requirements have been met.

### 3. Compositions:

Example of compositions for all dosage strengths are listed in Table 3-1 through Table 3-6

TABLE 3-1
Composition at 5:1 ratio for 250/50 mg Met/LAF, film coated tablets

| | Amount per | Weight per |
|---|---|---|
| Component | tablet (mg) | weight (%) |
| LAF237 | 50.0 | 15.3 |
| Metformin HCl | 250.0 | 76.3 |
| Hydroxypropyl cellulose (Klucel ® EXF) | 24.7* | 7.6 |
| Magnesium stearate | 2.9 | 0.9 |
| Total core weight | 328.0 | 100.0 |
| Film coating | | |
| Opadry premix** | 13.1 | 4.0 |
| Purified water, USP | q.s.^{a} | |
| Total film coated tablet weight | 341.0 | |
| LAF237 | 50.0 | 15.24 |
| Metformin HCl | 250.0 | 76.22 |
| Hydroxypropyl cellulose (Klucel ® EXF) | 24.75* | 7.6 |
| Magnesium stearate | 3.25 | 0.99 |
| Total core weight | 328.0 | 100.0 |
| Film coating | | |
| Opadry premix** | 12.0 | 3.53 |
| Purified water, USP | q.s.^{a} | |
| Total film coated tablet weight | 340.0 | |

^{a}Removed during processing.
*9% (w/w) calculated based on total quantity Metformin HCl and HPC.

TABLE 3-2
Composition at 10:1 ratio for 250/25 mg and
500/50 mg Met/LAF, film coated tablets

| | | 250/50 mg | | 500/50 mg |
|---|---|---|---|---|
| | 250/50 mg | weight per | 500/50 mg | weight per |
| | amount per | weight | amount per | weight |
| Component | tablet (mg) | (%) | tablet (mg) | (%) |
| LAF237 | 50.0 | 8.3 | 50.0 | 8.2 |
| Metformin HCl | 250.0 | 82.7 | 500.0 | 82.7 |
| Hydroxypropyl | 24.7* | 8.2 | 49.5* | 8.2 |
| cellulose (Klucel ® EXF) | | | | |
| Magnesium stearate | 2.7 | 0.9 | 5.4 | 0.9 |
| Total core weight | 302.0 | 100.0 | 605.0 | 100.0 |
| Film coating | | | | |
| Opadry premix** | 12.1 | 4.0 | 24.2 | 4.0 |
| Purified water, USP | q.s.^{a} | | q.s.^{a} | |
| Total film coated tablet weight | 315.0 | | 629.0 | |
| LAF237 | 50.0 | 15.24 | 50.0 | 8.25 |
| Metformin HCl | 250.0 | 76.22 | 500.0 | 82.51 |
| Hydroxypropyl | 24.75* | 7.55 | 49.5* | 8.17 |
| cellulose (Klucel ® EXF) | | | | |
| Magnesium stearate | 3.25 | 0.99 | 6.5 | 1.07 |
| Total core weight | 328.0 | 100.0 | 606.0 | 100.0 |
| Film coating | | | | |
| Opadry premix** | 12 | 3.52 | 18 | 2.89 |
| Purified water, USP | q.s.^{a} | | q.s.^{a} | |
| Total film coated tablet weight | 340.0 | | 624.0 | |

^{a}Removed during processing.
*9% (w/w) calculated based on total quantity of Metformin HCl and HPC.

TABLE 3-3
Composition at 17:1 ratio for Met/LAF 850/50 mg, film coated tablets

| | amount per | weight per |
|---|---|---|
| Component | tablet (mg) | weight (%) |
| LAF237 | 50.0 | 5.0 |
| Metformin HCl | 850.0 | 85.6 |
| Hydroxypropyl cellulose (Klucel ® EXF) | 84.1* | 8.5 |
| Magnesium stearate | 8.9 | 0.9 |
| Total core weight | 993.0 | 100.0 |
| Film coating | | |
| Opadry premix** | 39.7 | 4.0 |
| Purified water, USP | q.s.^{a} | |
| Total film coated tablet weight | 1033.0 | |
| LAF237 | 50.0 | 5.03 |
| Metformin HCl | 850.0 | 85.51 |
| Hydroxypropyl cellulose (Klucel ® EXF) | 84.15* | 8.47 |
| Magnesium stearate | 8.85 | 0.99 |
| Total core weight | 994.0 | 100.0 |
| Film coating | | |
| Opadry premix** | 26 | |
| Purified water, USP | q.s.^{a} | |
| Total film coated tablet weight | 1020.0 | |

^{a}Removed during processing.
*9% (w/w) calculated based on total quantity of Metformin HCl and HPC.

TABLE 3-4
Composition at 20:1 ratio for Met/LAF 500/25 mg
and 1000/50 mg, film coated tablets

| | 500/25 mg | 500/25 mg | | 1000/50 mg |
|---|---|---|---|---|
| | amount | weight per | 1000/50 mg | weight per |
| | per tablet | weight | amount per | weight |
| Component | (mg) | (%) | tablet (mg) | (%) |
| LAF237 | 25.0 | 4.3 | 50.0 | 4.3 |
| Metformin HCl | 500.0 | 86.3 | 1000.0 | 86.3 |
| Hydroxypropyl cellulose | 49.5* | 8.5 | 98.9* | 8.5 |
| (Klucel ® EXF) | | | | |
| Magnesium stearate | 5.2 | 0.9 | 10.4 | 0.9 |
| Total core weight | 580.0 | 100.0 | 1159.0 | 100.0 |
| Film coating | | | | |
| Opadry premix** | 23.2 | 4.0 | 46.4 | 4.0 |
| Purified water, USP | q.s.^{a} | | q.s.^{a} | |
| Total film coated tablet weight | 603.0 | | 1206.0 | |
| LAF237 | 25.0 | 4.31 | 50.0 | 4.31 |
| Metformin HCl | 500.0 | 86.21 | 1000.0 | 86.21 |
| Hydroxypropyl cellulose | 49.5* | 8.53 | 99* | 8.53 |
| (Klucel ® EXF) | | | | |
| Magnesium stearate | 5.5 | 0.95 | 11 | 0.95 |
| Total core weight | 580.0 | 100.0 | 1160.0 | 100.0 |
| Film coating | | | | |
| Opadry premix** | 18 | | 28 | 2.36 |
| Purified water, USP | q.s.^{a} | | q.s.^{a} | |
| Total film coated tablet weight | 598 | | 1188 | |

^{a}Removed during processing.
*9% (w/w) calculated based on total quantity of Metformin HCl and HPC.

TABLE 3-5
Composition at 34:1 ratio for Met/LAF 850/25 mg, film coated tablets

| | amount per | weight per |
|---|---|---|
| Component | tablet (mg) | weight (%) |
| LAF237 | 25.0 | 2.6 |
| Metformin HCl | 850.0 | 87.8 |
| Hydroxypropyl cellulose (Klucel ® EXF) | 84.1* | 8.7 |
| Magnesium stearate | 8.7 | 0.9 |
| Total core weight | 968.0 | 100.0 |
| Film coating | | |
| Opadry premix** | 38.7 | 4.0 |
| Purified water, USP | q.s.^{a} | |
| Total film coated tablet weight | 1006.0 | |

^{a}Removed during processing.
*9% (w/w) calculated based on total quantity of Metformin HCl and HPC.

TABLE 3-6
Composition at 40:1 ratio for Met/LAF 1000/25 mg, film coated tablets

| | amount per | weight per |
|---|---|---|
| Component | tablet (mg) | weight (%) |
| LAF237 | 25.0 | 2.2 |
| Metformin HCl | 1000.0 | 88.2 |
| Hydroxypropyl cellulose (Klucel ® | 98.9* | 8.7 |
| EXF) | | |
| Magnesium stearate | 10.2 | 0.9 |
| Total core weight | 1134.0 | 100.0 |
| Film coating | | |
| Opadry premix** | 45.4 | 4.0 |
| Purified water, USP | q.s.^{a} | |
| Total film coated tablet weight | 1179.0 | |

^{a}Removed during processing.
*9% (w/w) calculated based on total quantity of Metformin HCl and HPC.

### EXAMPLE 4

The tablets prepared in accordance with the above Description and examples can be tested as follows.

### Tablet Evaluation Methods

1. Average tablet weight. Twenty tablets are weighed on an analytical balance and the average tablet weight calculated.
2. Tablet breaking strength (kilo bond-kp). tablets are individually tested using a Schleuniger crushing strength tester, and the average breaking strength calculated.
3. Friability (% loss). 10 tablets, accurately weighed, are subjected to 10 minutes friability testing using a Roche Friabilator. The tablets are dedusted, reweighed, and the weight loss due to the friability is calculated as a percentage of the initial weight.
4. Dispersion Disintegration time DT (The test for dispersible tablets defined in the British Pharmacopoeia, 1988, Volume II, page 895-BP 1988). Tablets are tested in accordance to the above-defined BP test (without discs) for dispersible tablets. This utilizes water at a temperature of 19°-21° C.
5. Dispersion Quality. In accordance with the BP uniformity of dispersion test for dispersible tablets (BP 1988 Volume II page 895), two tablets are placed in 100 ml of water at 19°-21° C. and allowed to disperse.

### Granule Evaluation Methods

1. Loss on Drying (LOD). The residual moisture content of the granule (LOD) can be determined on a 3-4 g sample using a Computrac moisture analyser set at 90° C. operated in accordance with the manufacturer's procedure.
2. Weight Median Diameter (WMD). A 10 g sample of granule is sifted for 2 minutes at suitable pulse and sift amplitudes in an Allen Bradley sonic sifter in accordance with manufacturer's instructions. Sieves of 300 μm, 250 μm, 200 μm, 150 μm, 100 μm, 53 μm and 40 μm are used. The WMD is calculated from the cumulative percentage undersize size distribution using a computer program.

### EXAMPLE 5

### Improved Manufacturing Robustness

A preliminary compactibility assessment is carried out on a Carver press using different formulations.

Data demonstrate that our claimed compositions on being compressed with increasing levels of pressure (compression force) show well adapted tablet strength. In particular e.g. the herein described formulations have shown a good tablet strength and compactibility. With increasing pressure (compression force) our claimed formulations and selected ranges show a substantially useful increase in tablet strength.

A compactibility study (D. Becker, personal communication) is carried out on an instrumented Korsch single station press with force and displacement sensors on both upper and lower punches.

A clear indication is afforded from these data that LAF237 tablets are very likely to have poor tablet hardness/crushing strength unless diluted out using sufficient filler with excellent compactibility. However, our claimed formulations and selected ranges are particularly adapted to provide the required compactibility especially for the LAF237:metformin ratio of 1:5.

The results obtained show that convenient tablet hardness can be obtained if the metformin granules contain e.g. between 1 and 20% preferably between 3 and 13%, between 3 and 17.5% of a binder such as HPC.

### EXAMPLE 6

### Friability

Evaluation is carried out using a Manesty Betapress at 6 different settings: strain rate settings of 66-90 rpm (63,000-86,000 TPH) and force of 7.5-15 kN. The trials uses Flat-faced Beveled-edge (FFBE) tooling of 9 mm diameter for 250 mg tablets and 10 mm diameter for 310 mg tablets (other diameters are used depending on the weight of the tested tablet). Friability, Compression profile, Strain rate profile and Weight variation are the measured outcomes. Study design and the friability results obtained from the study are used to determine the variables (particle size distribution in the formulation, tablet weight, tablet thickness and weight, water content in the tablet etc) impacting the outcome of hardness. Our claimed formulations and selected ranges are particularly adapted to provide the required Friability.

Example—Tablets having a Metformin:LAF237 ratio of 20:1: The results show that tablets comprising LAF237+(metformin granules without binder) have around 0.8% friability, while tablets comprising LAF237+(metformin granules comprising 12% HPC) have less than 0.2% friability (at a compression force of 15 kN).

### EXAMPLE 7

### Mechanical Stress (Particle Size Distribution)

The material in the desired particle size range can be produced from any form of vildagliptin e.g. amorphous vildagliptin, by mechanical stress. This stress can be mediated by impact, shear or compression. In most commercially available grinding equipment a combination of these principles occurs. For vildagliptin preferably a mechanical impact or jet mill is used. The most preferable mechanical impact mill can be equipped with different kind of beaters, screens, liners or with pin plates. For our process preferably an impact mill with plate beater and a slit screen 5*2.5 cm is used. The impact speed should be variable between 20 and 100 m/s (as peripheral speed) to adapt to any batch to batch variation. In our case a peripheral speed of the beater of about 40-50 m/s is used.

1. A pharmaceutical composition comprising between 50 to 98% by weight on a dry weight basis of active ingredients, wherein the active ingredients consist of vildagliptin and metformin, or in each case a pharmaceutically acceptable salt thereof.

2. A tablet, comprising between 50 to 98%, by weight on a dry weight basis of active ingredients, wherein the active ingredients consist of vildagliptin and metformin, or in each case a pharmaceutically acceptable salt thereof.

3. (canceled)

4. The composition according to claim 1, comprising at least one pharmaceutically acceptable excipient.

5. The composition according to claim 1, wherein metformin is in the form of granules.

6. (canceled)

7. The composition or tablet according to claim 5, further comprising a binder.

8. The composition or tablet according to claim 7, wherein the binder is between 1 to 25% by weight.

9. (canceled)

10. (canceled)

11. A pharmaceutical composition comprising as active ingredients,
i) between 1.5 to 35% of a DPP-IV inhibitor, or a pharmaceutically acceptable salt thereof,
ii) between 65 to 98.5% of metformin or a pharmaceutically acceptable salt thereof,
and wherein metformin is in the form of granules and wherein the composition comprises between 1 to 25% of a binder.

12. The composition according to claim 11, comprising as active ingredients,
i) between 1.5 to 20% of vildagliptin, or a pharmaceutically acceptable salt thereof,
ii) between 80 to 98.5% of metformin or a pharmaceutically acceptable salt thereof,
and wherein metformin is in the form of granules and wherein the composition comprises between 1 to 25% of a binder.

13. (canceled)

14. The composition according to claim 7, wherein the binder is selected from starches; celluloses and derivatives thereof; sucrose; dextrose; corn syrup; polysaccharides; and gelatin.

15. The composition according to claim 7, wherein the binder is a cellulose or derivative thereof, selected from microcrystalline cellulose, hydroxypropyl cellulose, hydroxylethyl cellulose and hydroxylpropylmethyl cellulose.

16. (canceled)

17. The composition according to claim 4, wherein the pharmaceutically acceptable excipient is selected from binders, diluents, disintegrants, lubricants, solid fillers, glidants and carriers.

18. (canceled)

19. (canceled)

20. The composition or tablet according to claim 4, wherein the further pharmaceutically acceptable excipient is a lubricant.

21. The composition according to claim 20, comprising between 0.1% to 5%, by weight of the composition of a pharmaceutically acceptable lubricant.

22. The composition according to claim 20, wherein the lubricant is magnesium stearate.

23. (canceled)

24. (canceled)

25. (canceled)

26. (canceled)

27. (canceled)

28. (canceled)

29. The pharmaceutical composition according to claim 1, which is contained in a capsule or is in the form of a tablet, compressed tablet or directly compressed tablet.

30. (canceled)

31. (canceled)

32. (canceled)

33. A composition comprising;
i) between 25 mg and 100 mg of vildagliptin or a pharmaceutical salt thereof, or
ii) at least one pharmaceutically acceptable excipient.

34. (canceled)

35. (canceled)

36. The composition according to claim 1, comprising an additional active ingredient which is a sulfonylureas or a glitazone such as pioglitazone or rosiglitazone.

37. A process for preparing a pharmaceutical composition comprising vildagliptin and metformin or in any case a pharmaceutical salts thereof, which comprises:
i) granulating metformin and a binder,
ii) drying granules containing metformin and the binder,
iii) blending the vildagliptin DPP IV inhibitor, drug substance with the granules containing metformin and the binder; and
iv) optionally a lubricant e.g. magnesium stearate is blended with the mixture obtained on step iii),

38. A process for preparing a pharmaceutical tablet comprising vildagliptin and metformin or in any case a pharmaceutical salts thereof, which comprises;
i) granulating metformin and a binder,
ii) drying granules containing metformin and the binder,
iii) blending the vildagliptin, drug substance with the granules containing metformin and the binder,
iv) optionally blending a lubricant e.g. magnesium stearate is blended with the mixture obtained on step iii),
v) compressing the resulting blend to form tablets in unit dosage form.

39. (canceled)

40. (canceled)

41. The process according to claim 37, wherein at least one further pharmaceutically acceptable excipitent is added to the mixture to be blended during step i) or during step iii).

42. A process according to claim 41, wherein the further pharmaceutically acceptable excipient is a diluent or a disintegrant.

43. (canceled)

44. (canceled)

45. (canceled)

46. (canceled)

47. A process according to claim 37, wherein the binder is a cellulose or derivative thereof, selected from microcrystalline cellulose, hydroxypropyl cellulose, hydroxylethyl cellulose and hydroxylpropylmethyl cellulose.

48. (canceled)

49. (canceled)

50. (canceled)

51. (canceled)
